(19) United States
(12) Patent Application Publication
(10) Pub. No.: US 2012/0116156 A1
(43) Pub. Date: May 10, 2012

(54) METHOD AND APPARATUS FOR AUTOMATIC VERIFICATION OF ENDOTRACHEAL INTUBATION

(76) Inventor: Dror Lederman, Qiryat-Gat (IL)

(21) Appl. No.: 13/138,893

(22) PCT Filed: Apr. 27, 2010
(86) PCT No.: PCT/IB2010/051833

§ 371 Date: Jan. 23, 2012

Related U.S. Application Data
(62) Division of application No. 61/173,324, filed on Apr. 28, 2009.

(51) Int. Cl. A61B 1/267; A61B 1/04; A61B 1/07; A61M 1/00; A61B 1/307; A61B 1/273; A61B 1/233; A61M 16/04; A61B 1/00

(52) U.S. Cl. 600/109; 600/117

(57) ABSTRACT

A medical device includes a tube, at least one imaging sensor coupled to an endoscope in the tube, and a monitor application to monitor positioning of the tube in a medical patient by identifying expected anatomical features in images provided by the at least one sensor. A method for endotracheal intubation includes receiving imaging frames from a sensor located in an endotracheal tube inserted through a patient's mouth, and processing the imaging frames to identify a progression of anatomical features consistent with a proper placement of the endotracheal tube.

### CROSS-REFERENCE TO RELATED APPLICATIONS

This application claims benefit from U.S. Provisional Patent Application No. 61/173,324, filed Apr. 28, 2009, which is hereby incorporated in its entirety by reference.

### FIELD OF THE INVENTION

The present invention relates to medical intubation devices generally and to endotracheal intubation in particular.

### BACKGROUND OF THE INVENTION

Endotracheal tubes (ETTs) are known in the art. An ETT is a ventilating tube, typically made of plastic, which is introduced into a patient's trachea by a medical procedure known as “intubation”. ETTs ensure the patency (unblocked state) of the patient's airways by creating a safe artificial corridor for air to enter the lungs without obstruction.

To place the ETT in the trachea, an operator typically uses a tool, such as a laryngoscope, to move the tongue out of the way and illuminate the back of the throat. The laryngoscope is inserted into the patient's mouth and passes over the tongue, thusly visualizing the vocal cords. Once the vocal cords can be clearly seen, the operator can insert the ETT between the cords and into the trachea. Unfortunately, it's not always possible to obtain a clear view of the entrance to the trachea. In such cases there is a substantial risk of inserting the ETT into the esophagus instead of the trachea. Unintentional esophageal intubation can produce catastrophic results as the patient might be deprived of oxygen. If an ETT remains for too long in the esophagus, it may result in brain damage and even death.

Furthermore, even though the tube might be confirmed to be in an air conducting structure, it may not be correctly positioned. Over-insertion might position the tube past the bifurcation of the trachea (the “carina”). It might therefore be placed in the main left or right bronchus (the division of the trachea conducting air to the left/right lung respectively) thus ventilating only one lung (“endobronchial intubation”). Some patients might suffer oxygen deficiency while ventilated in only one lung. Prolonged single lung ventilation might also cause other serious pulmonary complications such as pneumonia.

In order to prevent the above complications a number of screening and monitoring devices are used in order to ensure proper ETT position. Listening to the lungs with a stethoscope (“auscultation”) in order to hear breath sounds is the most commonly used practice to confirm the correct positioning of an ETT. However, in thin patients breath sounds might be detected despite esophageal intubation. Conversely, in fat patients breath sounds might be so low that endobronchial intubation could go undetected.

Pulse oximetry and end-tidal CO2 (ETCO2) are generally considered among the best tests for tracheal ETT positioning. However, both of these methods rely on gas exchange to and from the lungs and are thus based on the assumption of proper perfusion and proper functioning of the lungs. Consequently, these methods cannot be used in various medical situations such as cardiac arrest and hypovolemic shock where carbon dioxide is not transported to the lungs. Furthermore, these methods cannot identify bronchial intubation.

A fiber-optic bronchoscope is among the most common tools used to aid in intubation procedures in hospitals when difficult intubation is anticipated. The bronchoscope is used to visualize the vocal cords. Unfortunately, this tool is costly, requires specific training, is cumbersome, and cannot be used in an out-of-hospital scenario. Consequently its use is very limited.

In recent years some visualization devices have been proposed as disclosed by several patents and scientific papers. There are several devices which are based on a miniature camera incorporated in the laryngoscope and an LCD screen. Since the camera is installed on the tip of the laryngoscope they enable “inserting” the operator's eye into the patient's throat. The operator can visualize the upper airway and vocal cords from a closer view angle while inserting the tube thus avoiding some of the tissues obscuring direct view of the vocal cords.

U.S. Pat. No. 5,400,771 to Wilk discloses an endotracheal intubation assembly and a related method. This invention is directed towards ultra sonic imaging of the trachea via the assembly.

Recently, an ETT with an embedded miniature camera and an LCD screen has been proposed as disclosed in WO2004/030527 issued to Gavriely. This invention provides continuous visualization of the airways to an expert for visual identification and verification of the anatomy and tube position.

### SUMMARY OF THE INVENTION

There is provided, in accordance with an embodiment of the invention, a medical device including a tube, at least one imaging sensor coupled to an endoscope in the tube, and a monitor application to monitor positioning of the tube in a medical patient by identifying expected anatomical features in images provided by the at least one sensor.

Further, in accordance with an embodiment of the invention, the monitor application includes means to perform at least one of reference comparison, statistical modeling, unsupervised clustering and ellipse detection.

Further, in accordance with an embodiment of the invention, the tube is an endotracheal intubation tube and the anatomical features are at least one of vocal cords, carina and trachea.

Still further, in accordance with an embodiment of the invention, the anatomical features are at least one of esophagus and bronchus.

Additionally, in accordance with an embodiment of the invention, the at least one imaging sensor is at least one of a camera and audio sensor.

Moreover, in accordance with an embodiment of the invention, the sensor and its associated electrical wiring are embedded in a wall of the tube.

Further, in accordance with an embodiment of the invention, the device also includes a flexible transparent sleeve attached to the tube, and means to insert the at least one imaging sensor into the sleeve, where the means are at least one of a separate endoscope and stylet to house the at least one imaging sensor and its associated electrical wiring.

Still further, in accordance with an embodiment of the invention, the device also includes an optic fiber conducting light from a lighting source located at a proximal end of the tube to a distal end of the tube.

Additionally, in accordance with an embodiment of the invention, the tube is appropriate for at least one of the following nasoenteric feeding, urine drainage, and coniotomy.

Moreover, in accordance with an embodiment of the invention, the device also includes a disposable adapter to hold the sensor and its associated wiring in place.

There is also provided, in accordance with an embodiment of the invention, a method for endotracheal intubation including receiving imaging frames from a sensor located in an endotracheal tube inserted through a patient's mouth, and processing the imaging frames to identify a progression of anatomical features consistent with a proper placement of the endotracheal tube.

Further, in accordance with an embodiment of the invention, the progression of anatomical features is at least one of a carina identified with high probability, and a carina identified with low probability recently preceded by an identification of vocal cords.

Still further, in accordance with an embodiment of the invention, the method also includes identifying a bronchial intubation, where the bronchial intubation is inferred from an unidentified anatomical feature recently proceeded by an identification of a carina.

Additionally, in accordance with an embodiment of the invention, the method also includes identifying a esophageal intubation, where the esophageal intubation is inferred from an unidentified anatomical feature that was not proceeded by an identification of a carina.

Moreover, in accordance with an embodiment of the invention, the method also includes indicating results of the processing to an operator of the tube.

Further, in accordance with an embodiment of the invention, the processing includes determining whether a distance from the sensor to an identified carina is consistent with a proper placement of the endotracheal tube.

Still further, in accordance with an embodiment of the invention, the determining includes performing segmentation and unsupervised clustering of the imaging frames depicting the identified carina, calculating a clusters area from the imaging frames, calculating a ratio between the clusters area and a total image area, and calculating the distance as a*r+b, where r is equal to the ratio and a and b are determined empirically based on a training database.

Additionally, in accordance with an embodiment of the invention, the method also includes identifying the anatomical features using at least one of reference comparison, statistical modeling, unsupervised clustering and ellipse detection.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic drawing of a novel apparatus for automotive verification of endotracheal intubation, designed and operative in accordance with an embodiment of the invention;

FIG. 2A is a schematic drawing of the apparatus of FIG. 1, designed and operative in accordance with another preferred embodiment of the invention;

FIG. 2B is a schematic drawing of a camera-embedded stylet to be used in the apparatuses of FIGS. 1 and 2;

FIG. 3 is a schematic drawing of a disposable adaptor to be used the apparatuses of FIGS. 1 and 2 to hold the stylet of FIG. 2B in place;

FIG. 4A is a block diagram of a classification algorithm to be used with the apparatuses of FIGS. 1 and 2;

FIGS. 4B-C are block diagrams of processing algorithms to be used with the apparatuses of FIGS. 1 and 2;

FIG. 5 is an exemplary image of the carina of a cow and a classification decision as determined by the apparatuses of FIGS. 1 and 2; and

FIG. 6 is another exemplary image of the carina of a cow as determined by an alternative embodiment of the apparatuses of FIGS. 1 and 2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the invention may be practiced without these specific details. In other instances, well-known methods, procedures, and components have not been described in detail so as not to obscure the invention.

As described in the Background, the prior art comprises a variety of tools and methods for the insertion of an ETT, verifying its correct placement and monitoring its operation over time. However, whether used individually or in combination, these tools and methods may suffer from a variety of drawbacks. For example, they are often expensive, cumbersome and/or may rely on the presence of an expert operator. Verification of correct placement may be unreliable or untimely, with possibly catastrophic results for the patient.

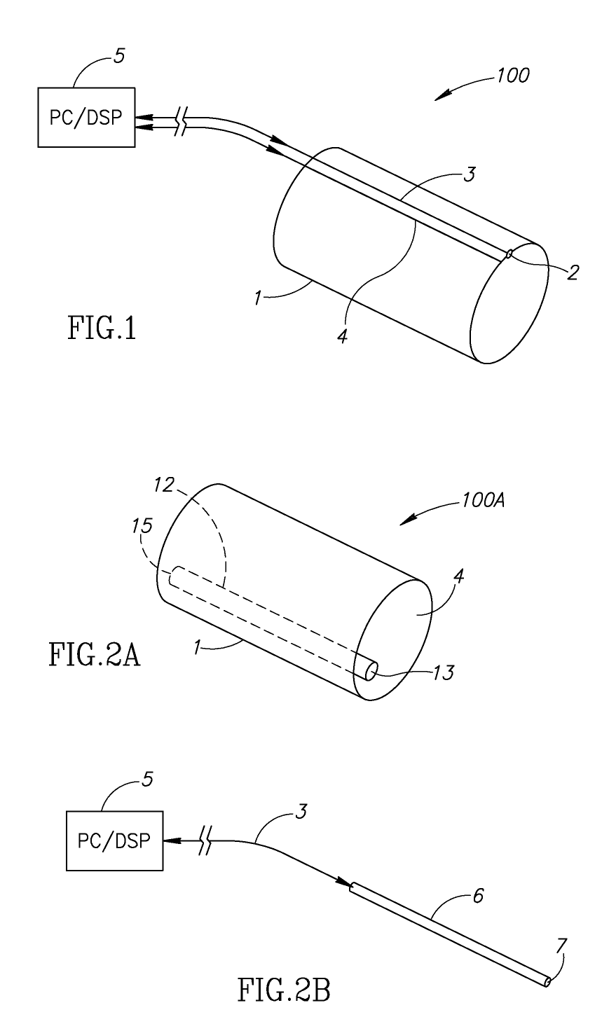

Applicants have realized that by adding visualizing capability and automatic placement verification functionality to an existing prior art ETT, a single apparatus may be used by a medical practitioner to insert the ETT, automatically verify its correct placement and monitor its operation over time. FIG. 1, to which reference is now made, illustrates a novel ETT insertion and automatic monitoring system 100, designed and operative in accordance with an embodiment of the invention.

System 100 may comprise a plastic endotracheal tube 1, similar in appearance and function to a prior art ETT. Miniature sensor 2, electric wires 3 and a wire-guided fiberscope 4 may be attached to the upper or lower wall of tube 1. Sensor 2 may be an audio or imaging sensor, such as a CCD, CMOS or any other imaging sensor. In accordance with an alternative embodiment of the invention, multiple miniature sensors may also be employed to provide both video and audio signals. Wire-guided fiberscope 4 may comprise, for example, a fiber-optic illumination or alternatively a LED. Sensor 2 may be connected via wires 3 to a computer or a Digital Signal Processor (DSP) card 5. DSP card 5 may comprise an integrated audio and video acquisition component, a miniature speaker an LCD screen and optionally several LEDs. It will be appreciated that the device may optionally include a small-size diameter tube or alternatively an endoscope to deliver air, oxygen or water in order to wash secretions and clean a camera sensor 2.

Video and/or audio signals obtained by sensors 2 may be transmitted through the electric wires to DSP 5, to be processed by monitor 8, a software application implemented on DSP 5. Alternatively, the signals may be transmitted using any type of wireless transmitter located at the tip of the tube and received by a receiver located on DSP 5.

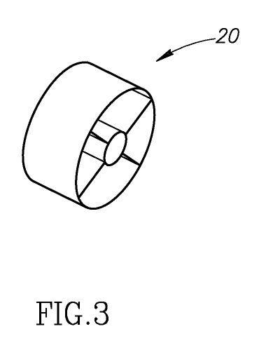

In accordance with an alternative embodiment of the invention, sensors 2, electric wires 3 and fiberscope 4 may be encased in a separate sleeve in order to facilitate their re-use and reduce the costs of system 100. Reference is now made to FIGS. 2A and 2B which together illustrate an option for implementing a non-disposable version of system 100. As shown in FIG. 2A, system 100A may comprise a plastic endotracheal tube 1 as in the previous embodiment. A flexible transparent sleeve 12 may be attached to tube 1, which may have an opening 13 and a closed distal tip 15. A stylet 6 as shown in FIG. 2B may be suitable for insertion in sleeve 12. Miniature camera 7 with a lighting element may be embedded in stylet 6, and connected via wires 3 to DSP 5. It will be appreciated that during operation, stylet 6 may be inserted through opening 13 in sleeve 12, such that miniature camera 7 may be placed in close proximity to distal tip 15.

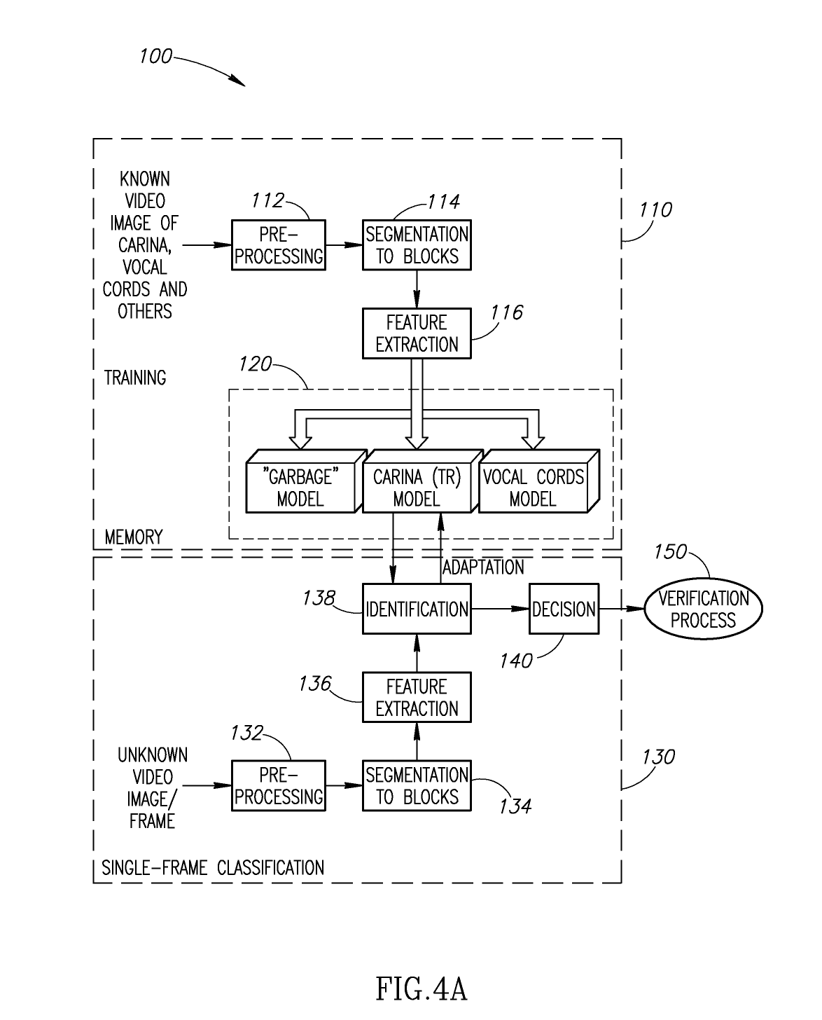

In accordance with another embodiment of the invention, stylet 6 may be used in conjunction with a prior art ETT. FIG. 3, to which reference is now made, illustrates a designated disposable adaptor 20 which may fit inside a standard-size endotracheal tube. Adapter 20 may fix stylet 6 in place during operation. It will be appreciated that using disposable adapter 20 it may reduce the cost of the device and enable the use of any standard-size endotracheal tube.

It will be appreciated that any of the above-mentioned embodiments may consist of only part of the sensors or electronic devices. For example, the invention may be implemented with only audio sensors, i.e. microphones and a speaker, embedded in endotracheal tube 1 or stylet 6. In such an example, monitoring and position verification may be performed based on analysis of the reflection of some audio signals transmitted by the speaker and received at the microphones located at the tip of endotracheal tube 1 and/or stylet 6. In such manner, costs reductions may be achieved since no imaging sensor and lighting element may be required.

Monitor 8 (FIG. 1) may perform an automatic position verification algorithm to validate the positioning of ETT 1. In accordance with an embodiment of the invention, a Hidden Markov Model (HMM) algorithm may be used by monitor 8 to classify images received from sensors 2. HMMs are known in the art and have been used extensively and successfully in many signal and image classification applications. The use of HMMs may allow different anatomical structures, in particular vocal cords and carina, to be easily represented and distinguished by different HMMs. Alternatively, the automatic position verification algorithm may be based on any other known pattern classifier or algorithm based on machine learning, such as support vector machines, neural networks, logistic regression, linear regression, Bayes classifier, etc. It will be appreciated that proprietary algorithms may also be used with the invention.

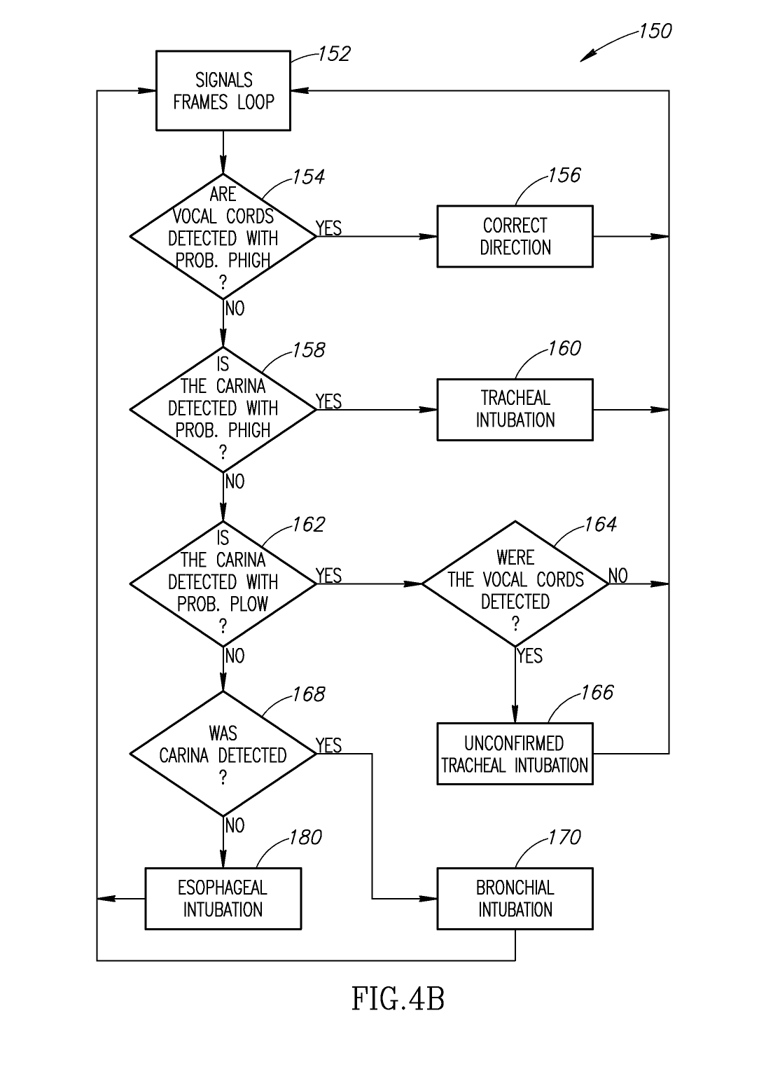

FIGS. 4A and 4B, to which reference is now made, illustrate the steps of an exemplary HMM based algorithm for the automatic position verification of images that may be received by monitor 8. Automatic position verification algorithm 100 comprises three main phases. A training phase 110, a single-frame classification phase 130 and a final position verification phase 150.

Training phase 110 (FIG. 4A) may be performed in a reduced and robust feature space representation of known images of relevant anatomical features such as, for example, the carina and vocal cords. Each image may be first pre-processed (step 112) in order to suppress background noise. Then, the image may be segmented (step 114) into overlapping blocks. Various features may then be extracted (step 116) from each block using, for example, calculations such as discrete-cosine transform (DCT). A description of the three main phases of the verification algorithm follows. The extracted models may be stored (step 120) as reference models for use during the single frame classification phase.

During single-frame classification phase 130, an image classifier may pre-process (step 132) video image frames, segment (step 134) them and extract (step 136) features from them as in steps 112, 114 and 116 of training phase 110. Each image may then be identified (step 138) in reference to the reference models stored in step 120. The probability of an image match may be generated for each one of the models. The probability may be calculated based on a maximum-likelihood criterion or alternatively based on any other scoring algorithm. The classification decision (step 140) for a particular image may be based on a calculated classification score. In some cases, a rejection policy may be applied to allow rejection of unrecognized images.

The classification decisions made for the last N frames may be input to verification process 150 (FIG. 4B). As will be described hereinbelow, process 150 may return at least the following possible results:

Vocal cords detected (correct direction);

Tracheal intubation confirmed;

Unconfirmed tracheal intubation;

Bronchial intubation detected; and

Esophageal intubation detected.

Identified images may be input (step 152) into a processing loop. If vocal cords are detected (step 154) with high probability, process 150 may return (step 156) a result of “correct direction” and return to step 152 to receive the next identified image.

If vocal cords are not detected (step 154) with high probability, the next step may be to query (step 158) whether the carina may have been detected with high probability. If the carina was detected with high probability, process 150 may return (step 160) a result of “tracheal intubation confirmed” and return to step 152 to receive the next identified image.

If the carina was not detected (step 158) with high probability, the next step may be to query (step 162) whether the carina may have been detected with low probability. If the carina was detected with low probability, previous results may be queried (step 164) to identify whether the vocal cords may have been recently identified in step 156. If the vocal cords were recently identified in step 156, then process 150 may return (a result of “unconfirmed tracheal intubation” and return to step 152 to receive the next identified image. Otherwise, if the vocal cords were not recently identified in step 156, then process may return to step 152 without returning a diagnostic result.

If the carina was not detected (step 162) with low probability, the next step may be to identify whether the carina may have been recently identified in steps 160 or 166. If the carina was recently identified, then process 150 may return (step 170) a result of “bronchial intubation” and return to step 152 to receive the next identified image. If the carina was not recently identified, then process 150 may return (step 180) a result of “esophageal intubation” and return to step 152 to receive the next identified image.

It will be appreciated that a user of system 100 may be prompted with appropriate visual and/or auditory cues regarding the results of process 150. For example, a green light may displayed as part of step 160; steps 156 and 166 may comprise a yellow light; step 170 may comprise an orange light; and step 180 may comprise a red light. It will be appreciated that system 100 may equipped with a display screen where the images and/or warning lights may be displayed as process 150 runs. Alternatively DSP 5 may comprise one or more LEDs to display indicators.

It should be noted that process 150 as depicted in FIG. 4B, as well as its parameters may be subject to minor changes and adjustments based on an appropriate database of images of human airways. For example, the values used to determine low and high probability (P_{low} and P_{high}) may be adjusted as necessary.

It will be appreciated that it may be important to generally determine an exact location of the ETT and its distance from the carina. FIG. 4C, to which reference is now made, illustrates a novel distance determination process 200, constructed and operative in accordance with an embodiment of the invention. Process 200 may be used by system 100 to analyze each and every carina detected image received as follows: System 100 may perform (step 210) segmentation and unsupervised clustering on the carina-detected image. The area of the clusters may then be calculated (step 220) prior to calculating (step 230) the ratio between the clusters area and the total image area. The distance may be calculated (step 240) as a linear function of the calculated ratio dist=a*ratio+b, where the parameters a and b may be determined empirically, based on a training database.

Alternatively or in addition, the distance may be obtained based on consecutives images analysis or based on the reflected audio signals acquired by sensors 2. ET tube 1 may generally be positioned about 2-5 cm above the carina in order to properly ventilate both lungs

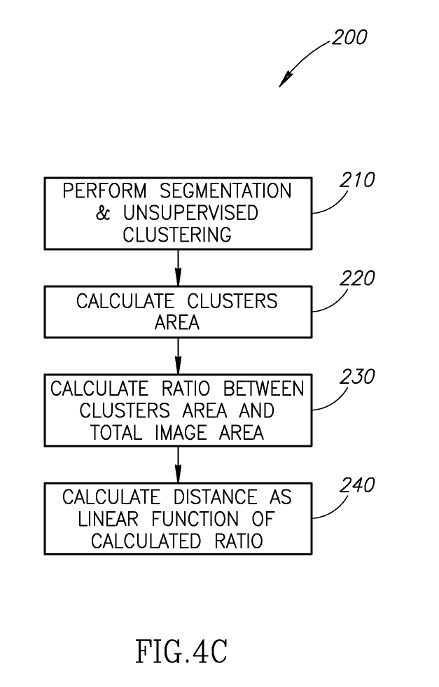

FIG. 5, to which reference is now made, illustrates an image of a cow's carina detected and verified by system 100. Tests results for system 100 using a cow's anatomy yield a 100% correct classification rate.

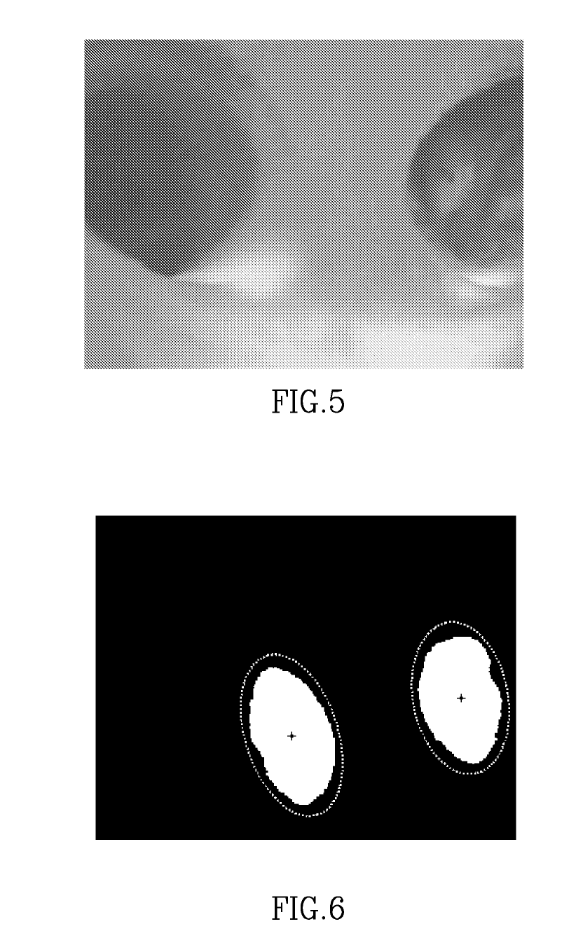

It will be appreciated that the detection of anatomical landmarks, such as, for example, the vocal cords and carina, etc. may not rely entirely on comparison with a reference image. In accordance with an alternative embodiment of the invention, unsupervised clustering methods, which are known in the art, and/or ellipse detection methods may be used separately or in combination for this purpose. The advantages of using such methods instead of reference comparison may be as follows: Such methods may enable real-time detection, which may result in faster processing. Also, as opposed to methods which are based on reference, these methods may not require a training phase to learn the reference images. Furthermore, such methods may be generally insensitive to physiological variability (i.e. adults, children, infants, etc.) and the effects of different imaging angles. Such methods may even improve on the overall accuracy attained by the implementations of the previous embodiments FIG. 6, to which reference is now made, illustrates an example of how such methods may be used to confirm correct tube position within the context of an alternative implementation of the process of FIG. 4B. In testing on cows, both unsupervised clustering and ellipse detection correctly classified 100% of the images.

In accordance with an alternative embodiment of the invention, endotracheal intubation may be verified based on reflected audio signals. As an alternative to analysis and classification of the images, or in addition to such classification, confirmation of correct endotracheal intubation may be based on analysis of reflected audio signals, acquired by microphones implemented as sensors 2.

A single tone may be generated and transmitted by DSP 5 through the miniature speaker embedded on ETT or on stylet. In case of an endotracheal intubation, the transmitted signal will encounter the carina, resulting in a relatively high energy of reflected signals. However, in case of esophageal intubation, the energy of the reflected signals, may be expected to be very low, due to the relatively low radiation resistance. Therefore, classification of the tube location may use a simplified version of the HMM classifier based on the following energy test:

$${\sum }_{n = 1}^{N} {x}^{2} ( t ) \begin{matrix}> \\ <\end{matrix} γ ,$$

where x may be the reflected signal acquired by microphones, and γ may be a predefined energy threshold. Alternatively, pattern recognition approaches may be employed to differentiate between carina-reflected pattern and esophageal-reflected pattern. It will be appreciated that using audio signals tests as an alternative to image classification may reduce costs. These tests may also be utilized in addition to image classification in order to improve classification rates by using verification based on both modalities—images analysis and reflected audio signals analysis; and to detect secretions, which might reduce the reliability of tube position verification based on image classification.

It will be appreciated that the invention may provide significant improvements over the prior art. For example, system 100 may provide a robust solution for patients of varying ages and body types. Since the relevant anatomy may differ from patient to patient, simple comparison between images may not be reliable and can not be used to determine correct tube position. The invention may be flexible enough to handle such differences.

It will similarly be appreciated that the invention provides an automated system and method for determining the location of the endotracheal tube. It may not be necessary for an expert operator to determine the efficacy of placement.

It will also be appreciated that the invention may indicate to the operator which images may be used during the process, thus enabling manual input as well.

It will likewise be appreciated that the invention includes a single system and method that may be used to both assist in the insertion of the endotracheal tube and monitor its correct placement. There may be no need for additional equipment to facilitate the process. Similarly, a hospital setting may not be required for operation.

Furthermore, it will also be appreciated that the invention may detect over insertion of the endotracheal tube, i.e. one lung intubation, and/or esophageal intubation. Accordingly, it may not just verify correct placement, but it may also provide specific warning in case of incorrect placement.

It will also be appreciated that the invention may be implemented within the context of any endoscopic procedure. For example, system 100 may be adapted for use for nasoenteric feeding and/or verification of automatic nasoenteric tube positioning. Similarly, system 100 may be adapted for use to drain urine from a patient's bladder and/or to automatically verify the placement of a urine or Foley catheter. System 100 may also be adapted for use in coniotomy procedures and/or for automatic verification of coniotomy tube placement.

In each such embodiment, endotracheal tube 1 may be replaced by a tube appropriate to the procedure being performed. Monitor 8 may also be adapted to process images received from sensors 2 in accordance with the anatomical features expected to be encountered during the given procedure. System 100 may be thusly adapted to process and verify anatomical images for any medical procedure requiring the placement of a tube within the body of a patient.

Unless specifically stated otherwise, as apparent from the preceding discussions, it is appreciated that, throughout the specification, discussions utilizing terms such as “processing,” “computing,” “calculating,” “determining,” or the like, refer to the action and/or processes of a computer, computing system, or similar electronic computing device that manipulates and/or transforms data represented as physical, such as electronic, quantities within the computing system's registers and/or memories into other data similarly represented as physical quantities within the computing system's memories, registers or other such information storage, transmission or display devices.

Embodiments of the invention may include apparatus for performing the operations herein. This apparatus may be specially constructed for the desired purposes, or it may comprise a general-purpose computer selectively activated or reconfigured by a computer program stored in the computer. Such a computer program may be stored in a computer readable storage medium, such as, but not limited to, any type of disk, including floppy disks, optical disks, magnetic-optical disks, read-only memories (ROMs), compact disc read-only memories (CD-ROMs), random access memories (RAMs), electrically programmable read-only memories (EPROMs), electrically erasable and programmable read only memories (EEPROMs), magnetic or optical cards, Flash memory, or any other type of media suitable for storing electronic instructions and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the invention are not described with reference to any particular programming language. It will be appreciated that a variety of programming languages may be used to implement the teachings of the invention as described herein.)

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A medical device comprising:
a tube;
at least one imaging sensor coupled to an endoscope in said tube; and
a monitor application to monitor positioning of said tube in a medical patient by identifying expected anatomical features in images provided by said at least one sensor.

1.1 The device according to claim 1 and wherein said monitor application comprises means to perform at least one of: reference comparison, statistical modeling, unsupervised clustering and ellipse detection.

2. The device according to claim 1 and wherein said tube is an endotracheal intubation tube and said anatomical features are at least one of vocal cords, carina and trachea.

3. The device according to claim 1 and wherein said anatomical features are at least one of esophagus and bronchus.

4. The device according to claim 1 and wherein said at least one imaging sensor is at least one of a camera and audio sensor.

5. The device according to claim 1 and wherein said sensor and its associated electrical wiring are embedded in a wall of said tube.

6. The device according to claim 1 and also comprising:
a flexible transparent sleeve attached to said tube; and
means to insert said at least one imaging sensor into said sleeve, wherein said means are at least one of a separate endoscope and stylet to house said at least one imaging sensor and its associated electrical wiring.

7. The device according to claim 1 and also comprising an optic fiber conducting light from a lighting source located at a proximal end of said tube to a distal end of said tube.

8. The device according to claim 1 and wherein said tube is appropriate for at least one of the following: nasoenteric feeding, urine drainage, and coniotomy.

9. The device according to claim 1 and also comprising a disposable adapter to hold said sensor and its associated wiring in place.

10. A method for endotracheal intubation comprising:
receiving video imaging frames from a sensor located in an endotracheal tube inserted through a patient's mouth; and
processing said video imaging frames to identify a progression of anatomical features consistent with a proper placement of said endotracheal tube.

11. The method according to claim 10 and wherein said progression of anatomical features is at least one of: a carina identified with high probability, and a carina identified with low probability recently proceeded by an identification of vocal cords.

12. The method according to claim 10 and also comprising identifying a bronchial intubation, wherein said bronchial intubation is inferred from an unidentified anatomical feature recently proceeded by an identification of a carina.

13. The method according to claim 10 and also comprising identifying an esophageal intubation, wherein said esophageal intubation is inferred from an unidentified anatomical feature that was not preceded by an identification of a carina.

14. The method according to claim 10 and also comprising indicating results of said processing to an operator of said tube.

15. The method according to claim 10 and wherein said processing comprises determining whether a distance from said sensor to an identified carina is consistent with a proper placement of said endotracheal tube.

16. The method according to claim 15 and wherein said determining comprises:
performing segmentation and unsupervised clustering of said imaging frames depicting said identified carina;
calculating a clusters area from said imaging frames;
calculating a ratio between said clusters area and a total image area; and
calculating said distance as a*r+b, wherein r is equal to said ratio and a and b are determined empirically based on a training database.

17. The method according to claim 10 and also comprising identifying said anatomical features using at least one of reference comparison, statistical modeling, unsupervised clustering and ellipse detection.
